(19) United States
(12) Patent Application Publication
(10) Pub. No.: US 2018/0174681 A1
(43) Pub. Date: Jun. 21, 2018

(54) LEAPING SEARCH ALGORITHM FOR SIMILAR SUB-SEQUENCES IN CHARACTER SEQUENCES AND APPLICATION THEREOF IN SEARCHING IN BIOLOGICAL SEQUENCE DATABASE

(71) Applicant: SUN YAT-SEN UNIVERSITY, Guangzhou (CN)

(72) Inventors: Yuesheng Xu, Guangzhou (CN); Ying Chen, Guangzhou (CN); Weicai Ye, Guangzhou (CN); Yongdong Zhang, Guangzhou (CN)

(21) Appl. No.: 15/740,038

(22) PCT Filed: Jun. 27, 2016
(86) PCT No.: PCT/CN2016/087300

§ 371 Date: Dec. 27, 2017

(30) Foreign Application Priority Data
Jun. 29, 2015 (CN) 201510373462.2

(51) Int. Cl. G16H 40/20; G06F 17/30

(52) U.S. Cl. CPC G16H 40/20; G06F 17/30463; G06F 17/3033; G06F 17/30339

(57) ABSTRACT

Disclosed is a leaping search algorithm for similar sub-sequences in character sequences and an application thereof in searching in a biological sequence database. The algorithm comprises: S0, constructing an FMD index and a lookup table for a database; S1, fetching, from the lookup table, a bi-interval of a sub-sequence with k characters in query sequences; S2, sequentially finding matching areas on the left of the k seed by using a backward search algorithm; S3, applying a forward search algorithm to an interval that has not been shrinked in step S2, to find matching areas on the right of the k seed; S4, checking whether the current detecting position is the end of the query sequence or not, and if yes, the algorithm ends, otherwise, proceeding to step S5; and S5, leaping forward w-k+1 positions from the current detecting position, and repeating steps S2 to S5. The lookup table proposed in the present invention features small memory space and a high access efficiency. According to the present invention, by combining the lookup table and an FMD index, all w seeds can be found quickly. In addition, the present invention has been successfully applied to biological sequence alignment.

### TECHNICAL FIELD

The present invention relates to the technical field of similar sub-sequence search in character sequences and character database searching, and in particular relates to a leaping search algorithm for similar sub-sequences in character sequences and an application thereof in searching in a biological sequence database.

### BACKGROUND

This algorithm is used for searching for similar sub-sequences in character sequences and achieving the purpose of rapidly retrieving similar sub-sequences by finding out seeds of the similar sub-sequences. Seed-based algorithm for similar character sub-sequence search has been widely used. BLAST and BWA algorithms commonly used in biological sequence analysis are representatives. Hereinafter, this algorithm will be described by taking biological sequence search as an example (but not limited to biological sequences). There are three types of existing solutions for searching for completely matching seeds having a length of at least w between a character sequence database and a query sequence.

The first type of algorithm is to construct a lookup table for the query sequence. The lookup table is a Hash table. Each entry is a linear linked list and records all positions of a sequence having a length of k in the query sequence. Such an algorithm then performs leaping scanning on the sequences in the database. Leaping scanning refers to detecting a sub-sequence having a length of k once every w-k+1 positions. The detection process includes finding out the positions of this sub-sequence in the query sequence by means of the lookup table, each position corresponding to a seed having a length of k, and then comparing the areas on the left and right of each k seed to check whether this k seed is contained in a w seed. Such a seed search algorithm is applied to MegaBLAST.

The second type of algorithm is to construct a lookup table for the database. This lookup table is also a Hash table. Each entry corresponds to a short sequence having a length of k. There are two types of typical solutions. The first type of solution, taking Indexed MegaBLAST as a representative, is to fetch a sub-sequence having a length of k from the database every w-k+1 positions and add the positions thereof into a corresponding Hash entry. The algorithm then detects all sub-sequences having a length of k in the query sequence to find out k seeds and then check whether these k seeds are contained in a w seed by means of solution I. The second type of solution, taking BLAT as a representative, is to fetch all non-overlapping sub-sequences having a length of k from the sequences in the database, record the positions thereof in corresponding Hash entries, and then check all sub-sequences having a length of w in the query sequence to find out a position linked list thereof in the Hash table, each position in the linked list corresponding to one w seed.

The third type of solution is to establish an FM index and or FMD index for the database and search for a maximum matching area having a length of at least w by means of this index. The maximum matching area refers to a completely matching area which cannot continue extending to the left and right. Sequence alignment software Cushaw2 adopts FM index to start from the first character of the query sequence and add one character from right in each step until the search result is a null set. The algorithm then starts from where the last step stops to continue the above process. Sequence alignment software BWA-MEM adopts FMD to search for the super maximum match. The super maximum match is also maximum match but the segment thereof on the query sequence cannot be covered by the segments of other maximum matches on the query sequence.

The above first type of solution is an initially proposed solution, which is also the most inefficient seed search solution in these three types of solutions. In the second type of solution, Indexed MegaBLAST may run very fast on a small database and short query sequence. However, on a large database and long query sequence, the lookup table will become very huge and the performance will drop suddenly and even less efficient than MegaBLAST. BLAT is not an accurate algorithm and cannot ensure to find out all w seeds. Although the performance is good, the third type of solution cannot ensure to find out all seeds having a length of at least w either, thus causing decreasing of final search accuracy.

### SUMMARY

The main object of the present invention is to overcome the disadvantages and shortcomings in the prior art and provide a leaping search algorithm for similar sub-sequences in character sequences and an application thereof in searching in a biological sequence database.

In order to achieve the above object, the present invention adopts the following technical solution:

The present invention provides a leaping search algorithm for similar sub-sequences in character sequences, comprising the steps of:

S0, constructing a lookup table atop an FMD index of a database, where the lookup table may be implemented in many ways, each entry corresponding to a short sequence having a length of k and saving a bi-interval obtained by searching for the short sequence in the FMD index;

S1, calculating a hash value of a k sub-sequence in a query sequence, and fetching, from the lookup table, a bi-interval of a seed having a length of k corresponding thereto;

S2, sequentially extending matching areas on the left of the k seed by using a backward search algorithm;

S3, applying a forward search algorithm to the interval that has not been shrinked in step S2, to find matching areas on the right of the k seed;

S4, checking whether the current detecting position is the end of the query sequence, and if yes, the algorithm ends, otherwise, proceeding to step S5; and

S5, leaping forward w-k+1 positions from the current detecting position, and repeating steps S2 to S5, where w is the length of the seed to be searched.

As a preferred technical solution, the FMD index is in particular as follows:

a sub-sequence with k characters is referred to as a “k sub-sequence”, a completely matching segment with w characters among the query sequences and the sequences in the database is referred to as a “w seed”, sequence p is searched in the FMD index of the database, the search result is represented in the form of a bi-interval, and the bi-interval is represented with three integers, given a bi-interval of a nucleotide sequence “P” and a character “a”, “a” being one element in a character table, a bi-interval of “aP” is obtained by the backward search algorithm; a bi-interval of “Pa” is obtained by the forward search algorithm, the element count of a of the bi-interval is referred to as the size of this interval, which represents the number of appearance of P in the database, and if the bi-interval of P is a null interval, it indicates that P is not in the database.

As a preferred technical solution, the memory space for the lookup table is small and will not increase as the database size increasing.

As a preferred technical solution, in step S2, during backward search, if the bi-interval is shrinked or is null, it indicates that some k seeds encounter mismatching character pairs during leftward extension, and for an interval that has not been shrinked, the algorithm also needs to find out matching portions on the right of the corresponding seeds thereof by means of the forward search algorithm to find out a seed having a length of at least w.

As a preferred technical solution, in step S3, during the forward search, if the search interval is null, it indicates that this query sequence is not in the database, otherwise, a bi-interval of w sub-sequence in the query sequence will be obtained and be output as a result.

As a preferred technical solution, in step S5, there is no need to detect all k sub-sequences in the query sequence, instead, it merely needs to perform detection once every w-k+1 positions.

As a preferred technical solution, in step S0, it is characterized in that FMD index and lookup table are combined to perform seed search, and the lookup table may have a plurality of implementations.

The present invention also provides an application of a leaping search algorithm for similar sub-sequences in character sequences in searching in a biological sequence database. If the processed character sequence is a biological sequence, the FMD index is constructed on a biological sequence database or a biological sequence search dataset, and the lookup table is constructed on this FMD index.

As a preferred technical solution, the biological sequence includes DNA, protein or RNA.

Compared to the prior art, the present invention has the following advantages and technical effects.

1. The difference between the lookup table proposed in the present invention from the existing linear linked list based lookup table lies in that the size of the linear linked list based lookup table will increase with the increasing of the database. In a large database, this lookup table will occupy a very large storage space, and a long linear linked list will also make the seed searching process time-consuming. Compared to the lookup table constructed on the basis of FM index, the lookup table proposed in the present invention saves bi-intervals of short sequences, and the lookup table constructed on the basis of FM index saves suffix array intervals.

2. Compared to the existing algorithm, the seed search algorithm proposed in the present invention has advantages of high accuracy and high efficiency. Some traditional seed search algorithms cannot find out all w seeds and cannot ensure the search accuracy. Some also adopt the leaping scanning method but have low performances in large databases and long query sequences and can only check whether the k seed is contained in a w seed one by one.

3. The seed search algorithm proposed in the present invention also adopts the leaping scanning method but it can check whether a batch of k seeds are contained in a w seed once such that it has very good performance advantages.

### BRIEF DESCRIPTION OF THE DRAWINGS

FIG. 1 is a flowchart of a leaping seed search algorithm combined with FMD index and lookup table in the present invention.

### DETAILED DESCRIPTION

Hereinafter, the present invention will be described in further detail in combination with embodiments and accompanying drawings, but the present invention is not limited to this.

### Embodiment

This embodiment takes biological sequence searching as an example. The present invention is applied to accelerating the megablast algorithm and achieved more than ten times speedup under the premise of keeping the same result. This embodiment mainly includes two parts: a lookup table combined with FMD index and a seed search algorithm. The construction of the lookup table is aimed at the disadvantages of the lookup table adopted in the second type of algorithms in the background. In these algorithms, each entry of the lookup table is a linear linked list such that the lookup table will occupy a very huge storage space in a large database. Some algorithms like BLAT index a part of short sequences in order to reduce the size of the lookup table, which will sacrifice the search accuracy. Each entry in the lookup table in the present invention saves a bi-interval. Each bi-interval merely needs to be represented with three numbers. Thus, the size of the lookup table is fixed and will not change as the size of the database increasing.

The seed search algorithm is aimed at the disadvantages in the above second and third algorithms. In these algorithms, Indexed MegaBLAST can find out all w seeds but is less efficient in a large database since the linear linked list will be very long and thus it requires to frequently check whether a k seed is contained in a w seed. Other algorithms adopt methods which sacrifice the accuracy in order to improve efficiency and they can merely find out a part of w seeds. The seed search algorithm in the present invention adopts the leaping scanning method in the first type of algorithm and can check whether a batch of k seeds are contained in a w seed. As a result, this algorithm has very high execution efficiency while being accurate.

FMD index is an abbreviation of bidirectional FM-index. FM is an abbreviation of the names of two authors Ferragina Paolo and Manzini Giovanni who proposed FM index.

Before introducing these two parts in detail, this embodiment will first describe information related to FMD index. A sub-sequence with k characters in a nucleotide sequence is referred to as a “k sub-sequence”. A completely matching segment with w characters among the query sequences and the sequences in the database is referred to as a “w seed”. Sequence P is searched in the FMD index of the database. The search result is represented in the form of a bi-interval. The bi-interval is represented with three numbers. Given a bi-interval of a nucleotide sequence “P” and a character “a” (“a” is one of A, C, G and T), a bi-interval of “aP” is obtained by the backward search algorithm; and a bi-interval of “Pa” is obtained by the forward search algorithm. The element count in the bi-interval is referred to as the size of this interval, which represents the number of appearance of P in the database. If the bi-interval of P is a null interval, it indicates that P is not in the database.

The basic flow of the present invention is as follows:

S0, constructing a lookup table atop an FMD index of a database, where the lookup table may be implemented in many ways (including but not limited to Hash table), each entry corresponding to a short sequence having a length of k and saving a bi-interval obtained by searching for the short sequence in the FMD index;

S1, calculating a hash value of a k sub-sequence in a query sequence, and fetching, from the lookup table, a bi-interval of a seed having a length of k corresponding thereto;

S2, sequentially extending matching areas on the left of the k seed by using a backward search algorithm;

S3, applying a forward search algorithm to an interval that has not been shrinked in step S2, to find matching areas on the right of the k seed;

S4, checking whether the current detecting position is the end of the query sequence, and if yes, the algorithm ends, otherwise, proceeding to step S5; and

S5, leaping forward w-k+1 positions from the current detecting position, and repeating steps S2 to S5, where w is the length of the seed to be searched.

The lookup table in the present invention is constructed on the basis of the FMD index of a nucleotide sequence database. It is also a Hash table. Each entry corresponds to a short sequence having a length of k and saves a bi-interval obtained by searching for the short sequence in the FMD index. The size of this lookup table is independent of the size of the database. Since each character can only be one of A, C, G and T, the lookup table has 4^{k} entries. This lookup table can be used to immediately obtain a bi-interval of the k sub-sequence in the query sequence.

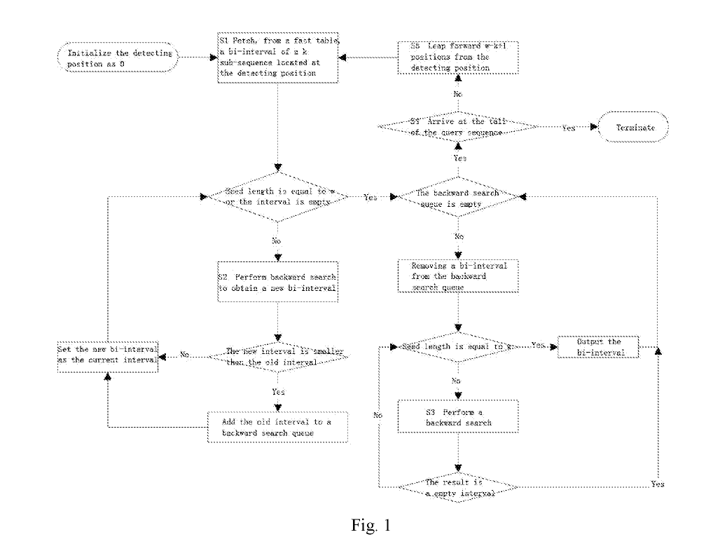

The second part of the algorithm is seed search algorithm, the flow of which is as shown in FIG. 1. This algorithm starts from the k sub-sequence at the most beginning of the query sequence to perform 5 main steps sequentially.

The first step of the algorithm is S1 in FIG. 1, which calculates the Hash value of the k sub-sequence and fetches a corresponding bi-interval thereof from the lookup table. The lookup table may not only realize leaping scanning but can also obtain the bi-interval of the k sub-sequence at one time without sequentially using a forward or backward search algorithm, which saves a large amount of time.

The second main step of the algorithm is S2 in FIG. 1. This step sequentially finds matching areas on the left of the k seed by using a backward search algorithm. During backward search, if the bi-interval is shrinked or is null, it indicates that some k seeds encounter mismatching character pairs during leftward extension. For an interval that has not been shrinked, the algorithm also needs to find a matching portion on the right of the seed corresponding thereto to find out a seed having a length of w.

The third main step of the algorithm is S3 in FIG. 1, which applies a forward search algorithm to the interval that has not been shrinked in step S2, to find matching areas on the right of the k seed. During forward search, if the search interval is null, it indicates that this query sequence is not in the database, otherwise, a bi-interval of w sub-sequence in the query sequence will be obtained and be output as a result.

The fourth step of the algorithm is part S4 in FIG. 1, which step checks whether the current detecting position is located at the tail of the query sequence. If yes, then the algorithm ends, otherwise, step 5 will be performed.

The fifth step of the algorithm is part S5 in FIG. 1, which leaping forwards w-k+1 positions from the current detecting position. Steps 2 to 5 are performed repeatedly, which is so-called leaping scanning. Leaping scanning does not no need to detect all k sub-sequences in the query sequence, instead, it merely needs to perform detection once every w-k+1 positions. This leaping scanning method has very high efficiency while ensuring that all w seeds can be found.

This embodiment also provides an application of a leaping search algorithm for identifying similar sub-sequences in a biological sequence database. If the processed character sequence is a biological sequence, the FMD index is constructed on a biological sequence database or a biological sequence search dataset, and the lookup table is constructed on this FMD index. The biological sequence includes but is not limited to DNA, protein or RNA. Other types of biological sequences are also suitable for the technical solution of the present invention.

The above embodiments are preferred embodiments of the present invention. However, the embodiments of the present invention are not limited by the above embodiments. Any other changes, modifications, replacements, combinations and simplifications, that are not departing from the spirit essence and principle of the present invention, shall all be regarded as equivalent substitutions and shall all be contained in the protection of scope of the present invention.

What is claimed is:

1. A leaping search algorithm for similar sub-sequences in character sequences, comprising the steps of:
S0, constructing a lookup table atop an FMD index of a database, each entry corresponding to a short sequence having a length of k and saving a bi-interval obtained by searching for the short sequence in the FMD index;
S1, calculating a hash value of the k sub-sequence in a query sequence, and fetching, from the lookup table, a bi-interval of a seed having a length of k corresponding thereto;
S2, sequentially extending matching areas on the left of the k seed by using a backward search algorithm;
S3, applying a forward search algorithm to the interval that has not been shrinked in step S2, to find matching areas on the right of the k seed;
S4, checking whether the current detecting position is the end of the query sequence or not, and if yes, the algorithm ends, otherwise, proceeding to step S5; and
S5, leaping forward w-k+1 positions from the current detecting position, and repeating steps S2 to S5, where w is the length of the seed to be searched.

2. The leaping search algorithm for similar sub-sequences in character sequences according to claim 1, wherein the FMD index is in particular as follows:
a sub-sequence with k characters is referred to as a “k sub-sequence”, a completely matching segment with w characters among the query sequences and the sequences in the database is referred to as a “w seed”, sequence p is searched in the FMD index of the database, the search result is represented in the form of a bi-interval, and the bi-interval is represented with three integers, given a bi-interval of a nucleotide sequence “P” and a character “a”, “a” being one element in a character table, a bi-interval of “aP” is obtained by the backward search algorithm; a bi-interval of “Pa” is obtained by the forward search algorithm, the element count of a bi-interval is referred to as the size of this interval, which represents the number of appearance of P in the database, and if the bi-interval of P is a null interval, it indicates that P is not in the database.

3. The leaping search algorithm for similar sub-sequences in character sequences according to claim 1, wherein the memory space for the lookup table is small and will not grow as the database size increasing.

4. The leaping search algorithm for similar sub-sequences in character sequences according to claim 1, wherein, in step S2, during backward search, if the bi-interval is shrinked or is null, it indicates that some k seeds encounter mismatching character pairs during leftward extension, and for an interval that has not been shrinked, the algorithm also needs to find out matching portions on the right of the corresponding seeds thereof by means of the forward search algorithm to find out a seed having a length of at least w.

5. The leaping search algorithm for similar sub-sequences in character sequences according to claim 1, wherein, in step S3, during the forward search, if the search interval is null, it indicates that this query sequence is not in the database, otherwise, a bi-interval of w sub-sequence in the query sequence will be obtained and be output as a result.

6. The leaping search algorithm for similar sub-sequences in character sequences according to claim 1, wherein, in step S5, there is no need to detect all k sub-sequences in the query sequence, instead, it merely needs to perform detection once every w-k+1 positions.

7. The leaping search algorithm for similar sub-sequences in character sequences according to claim 1, wherein the FMD index and lookup table are combined to perform seed search, and the lookup table may have a plurality of implementations.

8. An application of a leaping search algorithm for similar sub-sequences in character sequences in searching in a biological sequence database, wherein if the processed character sequence is a biological sequence, the FMD index is constructed on a biological sequence database or a biological sequence search dataset, and the lookup table is constructed on this FMD index.

9. The application of a leaping search algorithm for similar sub-sequences in character sequences in searching in a biological sequence database according to claim 8, wherein the biological sequence includes DNA, protein or RNA.
